(19) United States
(12) Patent Application Publication
(10) Pub. No.: US 2008/0172046 A1
(43) Pub. Date: Jul. 17, 2008

(54) Pulsed UV Laser Treatment of Recurring Skin Disorders

(76) Inventor: J. Peter Zimmer, Godfrey, IL (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/834,273

(22) Filed: Aug. 6, 2007

Related U.S. Application Data
(60) Provisional application No. 60/839,481, filed on Aug. 23, 2006.

(51) Int. Cl. A61B 18/20

(52) U.S. Cl. 606/9

(57) ABSTRACT

A method for treating and clearing dermatological conditions with a low energy emitting laser. One embodiment includes the treatment of psoriasis with a nitrogen based laser. When aimed through a hand piece by a physician, a pulsed UV laser having a wavelength of about 337 nM and pulse duration of between about 10 nS to 400 mS range, selectively targets the plaque of psoriasis. The beam is approximately 5 mm in diameter. This laser shot administers a therapeutic dose of UV light to the targeted area for the reduction and clearing of plaque associated with psoriasis without exposure of the surrounding skin. The method may be employed to treat psoriasis, eczema, vitiligo or other dermatological conditions.

### CROSS-REFERENCE TO RELATED APPLICATIONS

This application claims priority to provisional application Ser. No. 60/839,481, filed Aug. 23, 2006, which is incorporated herein by reference.

### FIELD OF THE INVENTION

This invention relates to the treatment of psoriasis and other recurring dermatological ailments disorders such as psoriasis, eczema, and vitiligo with very low power/energy laser wavelengths.

### BACKGROUND OF THE INVENTION

Dermatological diseases such as psoriasis, eczema, vitiligo and others are fairly prevalent. Psoriasis, for example, is a chronic, inflammatory skin condition which affects almost 3 percent of the U.S. population and about 1 to 3 percent of the worldwide population. Plaque psoriasis is the most common type of psoriasis. About 80% of people who develop psoriasis have plaque psoriasis, which appears as patches of raised, reddish skin covered by silvery-white scale. These patches, or plaques, frequently form on the elbows, knees, lower back, and scalp. However, the plaques can occur anywhere on the body.

Regardless of type, psoriasis usually causes discomfort. The skin often itches, and it may crack and bleed. In severe cases, the itching and discomfort may keep a person awake at night, and the pain can make everyday tasks difficult. Psoriasis is a chronic, meaning lifelong, condition because there is currently no cure. People often experience flares and remissions throughout their life. Controlling the signs and symptoms typically requires lifelong therapy.

Treatment depends on the severity and type of psoriasis. Some psoriasis is so mild that the person is unaware of the condition. A few develop such severe psoriasis that lesions cover most of the body and hospitalization is required. These represent the extremes. Most cases of psoriasis fall somewhere in between.

More than 4.5 million adults in the United States have been diagnosed with psoriasis, and approximately 150,000 new cases are diagnosed each year. An estimated 20% have moderate to severe psoriasis. Psoriasis occurs about equally in males and females. Recent studies show that there may be an ethnic link. It seems that psoriasis is most common in Caucasians and slightly less common in African Americans. Worldwide, psoriasis is most common in Scandinavia and other parts of northern Europe. It appears to be far less common among Asians and is rare in Native Americans. There also is a genetic component associated with psoriasis. Approximately one-third of people who develop psoriasis have at least one family member with the condition.

Research shows that the signs and symptoms of psoriasis usually appear between 15 and 35 years of age. About 75% develop psoriasis before age 40. However, it is possible to develop psoriasis at any age. After age 40, a peak onset period occurs between 50 and 60 years of age. About 1 in 10 people develop psoriasis during childhood, and psoriasis can begin in infancy. The earlier the psoriasis appears, the more likely it is to be widespread and recurrent.

Psoriatic arthritis develops in roughly one million people across the United States, and 5% to 10% experience some disability. Psoriatic arthritis usually first appears between 30 and 50 years of age—often months to years after skin lesions first occur. However, not everyone who develops psoriatic arthritis has psoriasis. About 30% of people who get psoriatic arthritis never develop the skin condition.

While scientists still do not fully know what causes psoriasis, research has significantly advanced our understanding. One important breakthrough began with the discovery that kidney-transplant recipients who had psoriasis experienced clearing when taking cyclosporine. Since cyclosporine is a potent immunosuppressive medication, this indicates that the immune system is involved.

Today, there are many different treatments to help control psoriasis. Some can be found over the counter at a drugstore, while others require a prescription from your doctor. Topical treatments—agents applied to the skin—are usually the first line of defense in treating psoriasis. Systemic drugs are prescription medications that affect the entire body, and are usually reserved for patients with moderate to severe psoriasis. Biologics are a type of systemic medication.

Many people choose to treat their psoriasis in nontraditional ways, including mind and body therapies, dietary supplements and sunlight. Phototherapy (UVB, PUVA and lasers) involves exposing the skin to wavelengths of ultraviolet light under medical supervision.

UVB treatment involves exposing the skin to an artificial UVB light source for a set length of time on a regular schedule, either under a doctor's direction in a medical setting or with a home unit purchased with a doctor's prescription.

PUVA is an acronym for psoralen (a light-sensitizing medication) combined with exposure to ultraviolet light A (UVA). UVA, like UVB, is found in sunlight. By itself, however, UVA is not usually used to clear psoriasis. It is relatively ineffective unless used with a light-sensitizing medication such as psoralen.

Sunlight has been known to decrease the intensity and occurrence of these psoriatic outbreaks. The UVB and PUVA treatment regimens are based on this knowledge.

In 1981 Parrish and Jaenicke studied the response of psoriasis clearing to different wavelengths of light and concluded that wavelengths of 290-313 would clear the skin of these lesions. The source for this study was a TL-01 Narrowband lamp made by Philips. For years this study was left unquestioned until Farr and Diffey in a letter to the editor of Photodermatology, PhotoImmunology and Photomedicine pointed out:

1 The light source in question only produced 5 wavelengths that were studied and that those were very limiting.

2 Only 4 patients were studied to make this conclusion.

Parrish J A, Jaenicke K F, Action spectrum for Healing of Psoriasis, Invest Dermatology, 1981; 76: 359-362)

Farr and Diffey point out that the specific wavelengths may not have the special healing properties and we should investigate this phenomenon further. (P M Farr, B L Diffey, Letter to the editor on Action Spectrum for Healing of Psoriasis, Photodermatology, PhotoImmunology, & Photomedicine 2006;22:52).

The excimer laser was invented in 1971 in Moscow by Nikolai Basov as a deep UV laser system. The current laser for psoriasis operates in the wavelength of 308 nM which falls into the favorable specifics of the 1981 study. The excimer laser however is very large, powerful, very expensive to purchase and very expensive to maintain. It is also very dangerous to use. In fact, research in this field is directed to the use of ever more powerful lasers. For example, lasers used in this field of medicine have energy outputs up to 8 millijoules per pulse.

### SUMMARY OF THE INVENTION

One aspect of the invention provides for the use of a relatively low power laser to treat dermatological conditions or disorders. The dermatological condition can be any recurring or non-recurring condition affecting the skin, including but not limited to psoriasis, eczema and vitiligo. One example is the use of a nitrogen laser to treat psoriasis. Another aspect of the invention provides for the application of energy at wavelength of 337 nM in the treatment of psoriasis and another aspect provides for the use of very low power/energy laser wavelengths for treatment of psoriasis and other dermatological conditions or disorders. These treatments may be provided in a physician's office or the patient's location or both.

One aspect of the invention is to provide a treatment for dermatological conditions at a fraction of the cost, at a fraction of the power required by known methods. Another aspect of the invention is to provide a very safe alternative to high power lasers, and drugs. Another aspect of the invention is to provide a treatment suitable for home therapy.

### BRIEF DESCRIPTION OF THE DRAWINGS

FIG. 1 is a diagrammatic view of a nitrogen laser device of the present invention;

FIG. 2 is another diagrammatic view of thereof; and

FIG. 3 is a diagrammatic view of a front panel of the nitrogen laser device of FIGS. 1 and 2.

### DETAILED DESCRIPTION OF ONE ASPECT OF THE INVENTION

One aspect of the present invention employs a low energy emitting laser to treat dermatological conditions. One embodiment of a laser employed in the present invention has a maximum output of 300 microjoules. In one aspect of the invention the low energy emitting laser is a nitrogen laser. In contrast to an excimer laser, a nitrogen laser is very inexpensive to build, maintain and operate. The laser is compact and can be constructed in a desktop or tabletop unit or a handheld unit. The laser may have an umbilical to attach two or more components together or could be wireless. The laser itself can be housed in main unit or in a hand piece.

In contrast to other sources of UV light treatment, the laser of the present invention can be more finely controlled to pin point treatment and avoid unnecessary exposure of surrounding tissues to UV energy. The laser could be controlled remotely. For example, the unit housed in the location of the patient, a satellite office or clinic could be controlled from a remote location by a medical practitioner through the use of a computer or through telephonic transmission so that the practitioner can control the treatments. The treating medical practitioner could remotely unlock the laser for a specific treatment through a computer which could be networked into the Internet along with the remotely controlled laser. Authorization codes could assure that the correct laser is used to treat the correct patient.

The monitoring of the laser treatment output can be monitored internally by the unit and allowed to emit laser energy only when the device meets all of its predetermined parameters thereby limiting any extraneous emissions that may be damaging to the patient, third persons of the environment.

In general the preferred nitrogen laser can produce wavelengths at about 337 nM. However, the invention contemplates the use of any laser that provides energy within the UV spectrum that is effective for treating the target dermatological disease. The UV wavelength, for example, should be effective to break the protein bonds within the RNA of healthy tissue, for example T-cells.

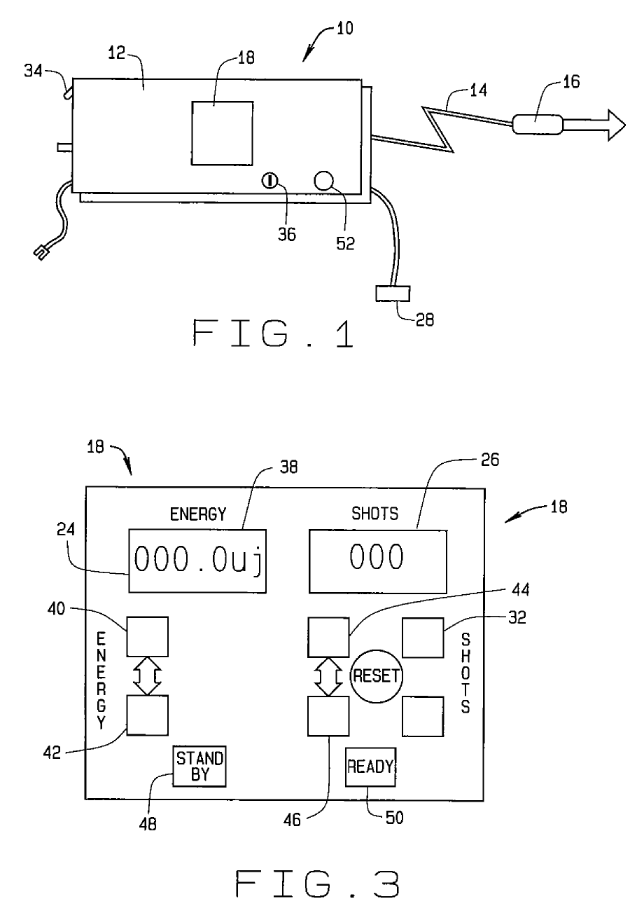

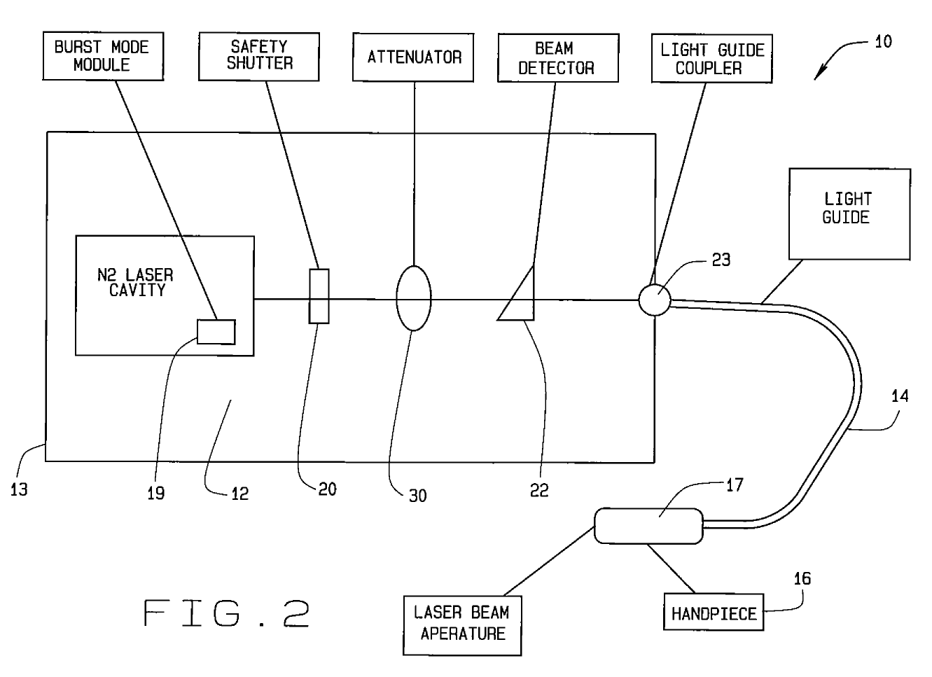

Referring to FIG. 1, one example of a nitrogen laser device that may be employed in the present invention preferably, but not necessarily, is a table top or freestanding unit, indicated generally by reference number 10, which is suitable for a physician's office or even the patient's location. It operates on standard electrical current available at the location it is placed. No external cooling is needed as it preferably is internally air (fan) cooled. Unit 10 includes a laser 12 which, in one embodiment, is a nitrogen laser capable of producing a spectral wavelength of about 337 nM, a pulse width between about 10 nS to 400 nS with a fast duty cycle to allow for a burst of pulses if selected. One example is a nitrogen laser with an output of about 17 micro joules and a pulse length of about 4 nano-seconds and a wavelength of about 337 nM. This laser can be enclosed in a light-tight case 13. The case may include, but is not limited to:

The laser 12:

A fiber optic 14 light guide for beam delivery with a beam delivery device, e.g. hand piece 16 with a laser beam aperture 17 that provides for an adjustable treatment spot size;

A control panel 18;

A burst mode module 19;

A safety shutter 20;

A beam detector 22;

A light guide coupler 23 for coupling the laser to the fiber optic

Energy monitor 24;

A device to count the number of treatment shots 26:

A foot pedal 28 to release the pulse from the beam delivery device 16 controlled with;

A beam attenuation mechanism 30 to allow for varying the amount of energy selected;

A mechanism 19 to allow for the firing of multiple shots with one press of the foot pedal 28.

The nitrogen laser 12 can be enclosed within the main case, as shown, and deliver its energy through the fiber optic 14 light guide or an articulated arm or waveguide or it can be enclosed within the hand piece 16 held by the physician. In a preferred aspect of the invention, the fiber optic 14 is matched exactly to the wavelength and the device through an infrared lockout system. The laser may include an optional aiming laser. The laser could be powered by 110 VAC through a conventional power cord 26 or 220 VAC where appropriate. The laser may include a battery back-up or rechargeable power source.

Although the basic configuration of laser unit 10 is described above for illustrative purposes, it will be appreciated that a laser including any or all of the necessary components to perform the treatment method described herein is intended to fall within the scope of the invention.

### Operation of the Device

Referring to FIG. 3, when a suitable patient presents for treatment, one aspect of a laser treatment procedure is as follows:

Enable power with Circuit Breaker (CB1) 34 on rear panel.

Unlock with the key switch (KS1) 36 on front panel.

The laser will then go through a self test for proper operation and then offer a “go/no go” indication in the displays; Energy display (ED) 38, Shot counter display (SC) 26. The display will signal the successful pass of the start up test.

The operator then selects the amount of energy by depressing button (LEI) 40 for increasing energy or button (LED) 42 for decreasing energy while noting the energy displayed on ED 38.

The operator then chooses the number of pulses per second that is desired by pressing button (PI) 44 to increase the number of shots of button (PD) 46 to decrease the number of shots while noting the display on SC 26.

The operator will determine the spot size by selecting the appropriate aperture 17 setting on the laser hand piece 16.

The laser will now indicate that it is in standby with indicator (LSI) 48 illuminating.

When the operator is satisfied that the laser, patient and physician is ready, the ready button (LRB) 50 is pressed switching the laser from standby to ready as indicated by the green Ready Indicator (LRI) 50.

As a final check, the operator must press Laser On Switch (LOS1 52) to allow the footswitch (FS1) 28 to function.

The physician can now treat the target area of the patient by holding the laser hand piece 16 so that its tip is above or just touching the patient's skin at the targeted area. The physician depresses the footswitch 28 and the laser indicates a treatment pulse with each “chirp” of the audible indicator and by the incrementing of the shot counter. In the illustrated aspect of the invention, the laser energy delivered to the treatment area is low dose and has a spectral wavelength of about 337 nM, a pulse width between about 10 nS to 400 nS.

At the conclusion of the treatment, the operator records the total number of shots and the total laser energy used to treat that specific patient for their permanent record. The laser may now be turned off with the key switch 36.

In another aspect of the invention, the laser is equipped with an appropriately programmed microprocessor (not shown) that accepts patient data either through keyboard input in the treatment room or office network input. The laser device will output the treatment parameters and significant data at the conclusion of treatment to the computer network for record keeping and billing purposes.

The device and method of the present invention allows for the administration of low dose UV radiation to the treatment area while leaving surrounding areas clear of the UV radiation.

The laser may be combined with a therapeutic drug, either systemic or topical. The laser may be used to activate or trigger the therapeutic drug. The laser may be used as a Photo Dynamic Therapy System (PDT).

These treatments, due to the low dose, may be provided in a physician's office or at the patient's location.

### Case Study and Dosimetry

### Subject:

The subject of the study was a 53 year old Caucasian male with a history of severe sudden onset psoriasis preceded by a diagnosis of a streptococcal throat infection in 1998. The infection was successfully treated with antibiotics and the psoriasis was treated with betamethasone and tar products. The psoriasis has been under control since treatment with the exception of one area of psoriatic plaque below the left elbow. The area of plaque measures approximately 2 cm by 3 cm with an irregular border. Upon severe flare-ups of the area, the subject generally made one application of a topical treatment of Betamethasone (0.05%) as prescribed by his dermatologist. No treatment with this drug was used for 2 months before the laser treatment described herein.

### Materials and Equipment:

A nitrogen laser manufactured by LSI (Newport Corporation) with an output of 17 micro joules and a pulse length of 4 nano-seconds and a wavelength of 337 nanometers was used. The beam area is approximately 0.5 cm in diameter. The laser has an adjustable output pulse train from 1 pulse per second to 20 pulses per second.

### Procedure:

The targeted area was cleaned with isopropyl alcohol prep and dried. The subject was given wavelength specific (UV) laser safely goggles to wear for eye protection. The laser was set for a pulse train of 10 pulses per second. The area treated was placed approximately 2 centimeters from the laser aperture. The laser was turned on, the laser aperture opened and the area was then treated for approximately 10 seconds by constantly moving the targeted area around so that the treatment area was given an even exposure of the laser energy.

Approximately 100 shots of a 337 nM laser pulses were used at 4 nS per pulse at 17 Micro joules per pulse.

### Results:

No redness or swelling was noted during or immediately following the procedure. No adverse effects were noted. This treatment took place approximately 2 pm and the following day the subject noted that the plaque formation that had normally been associated with the targeted area was missing. Subsequent inspection of that area over the next 7 days showed improvement. After the seventh day the area was still red, but the plaque had cleared entirely. A slight redness remains but there is no plaque or itching associated with the treatment area.

### CONCLUSION

Although the excimer laser has been proven that it clears psoriatic plaque within 2- to 8 treatments, the nitrogen laser cleared the subject's psoriatic plaque with one treatment.

It will be appreciated that the laser and method of the present invention may be altered or modified without departing from the scope of the invention. The invention is intended to include all types of nitrogen laser devices and all treatment protocols that result in an amelioration of the symptoms of psoriasis, including different pulse settings, placement of the laser beam, duration of treatment and so forth.

Although the foregoing description of the aspects of the invention, including the case study, was directed to a representative example of the invention, i.e. the treatment of psoriasis, it will be appreciated that the scope of the invention includes the use of a low energy laser, for example a Nitrogen laser of the type described, to treat any dermatological condition that is responsive to the low energy treatment including, but not limited to, psoriasis, eczema, vitiligo and the like.

Therefore, the written description, drawings, and case study are intended to be illustrative of the broader aspects of the invention, including the best mode of working the invention presently known to the inventor, and should not be relied on to construe the appended claims in any limiting manner.

I claim:

1. The treatment of dermatological conditions comprising treatment with a low energy emitting laser.

2. The treatment of claim 1 wherein the low energy emitting laser is a nitrogen based pulsed UV laser system.

3. The treatment of claim 1 further comprising delivery of a laser beam through a component selected from the group of components consisting of a fiber optic, a waveguide, an articulated arm and a hand piece.

4. The treatment of claim 1 further wherein the low energy is delivered in pulses adjustable between about 10 microjoules and about 200 micro joules

5. The treatment of claim 1 further comprising a laser pulse width between about 10 nS and about 400 nS.

6. The treatment of claim 1 further comprising multiple pulse releases with a burst mode.

7. The treatment of claim 1 wherein the laser can produce wavelengths at about 337 nM.

8. The treatment of claim 1 further comprising a targeted area of pulse delivery of low energy emitting laser energy of approximately 5 mm.

9. A method of destroying plaque developed in association with psoriasis comprising using a low energy emitting nitrogen laser to destroy said plaque at a treatment area of an individual's skin.

10. The method of claim 9 further comprising administering low dose UV radiation with the low energy emitting nitrogen laser to the treatment area of the skin while leaving surrounding areas of the skin clear of the UV radiation.

11. The treatment of claim 9 wherein the administration of the low dose UV radiation to treatment area is performed one time.

12. The treatment of a dermatological condition comprising treatment with a low energy emitting laser,
wherein the low energy emitting laser is a nitrogen based pulsed UV laser system;
wherein delivery of a laser beam through a component selected from the group of components consisting of a fiber optic, a waveguide, an articulated arm and a hand piece; and
wherein the low energy is delivered in pulses adjustable between about 10 microjoules and about 200 micro joules with a laser pulse width between about 10 nS and about 400 nS.

13. The treatment of claim 12 wherein the dermatological condition is selected from a group of dermatological conditions consisting of psoriasis, eczema and vitiligo.

14. The treatment of claim 12 wherein the laser produces wavelengths at about 337 nM.
